(19) United States
(12) Patent Application Publication
(10) Pub. No.: US 2012/0259301 A1
(43) Pub. Date: Oct. 11, 2012

(54) Ostomy skin barrier application method and device

(76) Inventors: John E. Polnik, Kingman, AZ (US); John N. Polnik, Seattle, WA (US)

(21) Appl. No.: 13/066,186

(22) Filed: Apr. 8, 2011

(51) Int. Cl. A61F 5/449

(52) U.S. Cl. 604/344

(57) ABSTRACT

A method and applicator for adhesively affixing an ostomy adapter to the skin of a user. Under ordinary circumstances an ostomy adapter includes an adhesively treated skin barrier on its underside which is affixed by uneven manual pressure, leading to adhesive seals that can leak, are unsanitary and uncomfortable. An applicator formed as two concentric annular cylinders, with a smaller diameter inner cylinder moving linearly within the larger outer cylinder, is able to apply a uniform pressure simultaneously along the adhesive seal between a skin barrier and the skin itself. The inner cylinder engages a hole in the skin barrier and both cylinders create a uniform downward pressure.

### BACKGROUND OF THE INVENTION

1. Field of the Invention

This invention relates generally to adhesive barriers that are applied between the skin surrounding a stomal opening and an ostomy apparatus including an ostomy adapter and a bag or pouch for receiving human waste. In particular it relates to an application device for attaching such an adhesive barrier easily, safely and with sanitary effectiveness by the user of such an ostomy apparatus.

2. Description of the Related Art

Many diseases of the intestinal and urinary system, such as ulcerative colitis, Crohn's disease and cancer are treated surgically by removal of a portion of the intestines or bladder. When such surgical removal occurs, the elimination of bodily waste is subsequently achieved through a newly formed opening, called a stoma, in an appropriate portion of the abdominal wall. The stoma is produced by a process called an ostomy, more specifically a colostomy or urostomy depending upon the particular anatomical region involved.

When waste is eliminated through the stoma, it is typically captured in a bag or pouch called an ostomy bag. The ostomy bag is removably attached to an ostomy adapter that fits snugly against the outer abdominal surface and is attached adhesively to the skin. The combined adapter and bag is generally referred to as an ostomy apparatus. Hereinafter we will refer to the portion of the apparatus that becomes affixed to the body as the ostomy adapter.

The front surface of the ostomy adapter includes a patterned portion that attaches to a disposable bag or pouch, the ostomy bag. The adapter has a central circular opening that fits over the stoma and that accepts the opening of the ostomy bag. The back surface of the adapter, which is in contact with the skin, includes an adhesive layer, or “skin barrier,” which is typically an adhesive coating over a plastic film, that forms an annular region around the central opening. The ostomy adapter is pressed against the skin of the user so that the adhesive portion of the skin barrier cures properly (eg., no air pockets or fluids should be trapped between the skin and the adhesive) and makes a firm and uniform seal that is strong enough to hold the adapter, the bag and its contents in place for a period of several days. In addition to its strength, the seal must be sanitary, free of wrinkles, conformable locally to the body shape and, generally, comfortable to wear. Such seals and adapters are well known in the prior art and their structure and method of use is described, for example, in Steer (U.S. Pat. No. 4,701,169), Mills et al. (U.S. Pat. No. 6,863,663) and also in Axelsson et al. (U.S. Pat. No. 7,699,824).

Needless to say, the variations in body type, including skin surfaces, abdominal wall shape, subcutaneous fat content, motion-induced shape variations, etc., make it difficult for any one size and shape ostomy adapter to fit all wearers. This fact, alone, is a good indication of the difficulty in applying the ostomy adapter so that it makes a good seal against the body. In addition to these obvious structural difficulties, it is easily recognized that many wearers of ostomy apparatuses may be physically debilitated, making the adapter difficult to apply correctly, even on the assumption that a correct fit is obtainable.

One commonly used adapter system is marketed by ConvaTec, a division of Bristol-Myers Squibb, under the trademark Esteem™ Synergy. The back surface of the adapter is a skin barrier that provides an adhesive surface to form a sanitary seal between the adapter and the skin. It is germane to the objects of this invention to briefly indicate the set of steps on a single illustrated sheet that accompanies the product. Note that these steps are written here purely as examples of the manipulations required by the user and it is noted that not all ostomy products would provide the same steps. While the steps and corresponding illustrations provided by the manufacturer are clear, it is to be remembered that these steps must be followed precisely by a possibly elderly or debilitated person while applying the device to their own bodies. The exemplary steps are as follows:

1. Measure stoma

2. Cut to appropriate size

3. A “transparent landing zone” is illustrated

4. Remove release paper (covering of adhesive)

5. Apply to skin (hands here shown making circular motions)

6. Remove release paper (covering of front surface)

7. Align pouch with the landing zone

8. Align pouch using an upward rolling motion

Of all these steps, one of the most important to perform correctly is step 5, where illustrated hands are shown pressing the adhesive backing of the skin barrier against the skin surrounding the stoma, while, at the same time, the user is being sure that the opening in the adapter remains properly aligned with the stoma. A method and device for assuring the proper completion of this step is an object of the present invention.

The inventors have discovered, through personal experience, that the process of correctly applying the adhesive backing of the skin barrier so that if forms a firm, safe, secure, comfortable and sanitary seal is difficult to achieve. It is the object of this invention, therefore, to provide a mechanical applicator that can be placed over the outer surface of the adapter and that will enable the user to produce such a secure, safe, comfortable and sanitary seal using a simple manual process applied in a simple and repeatable manner. It goes without saying that such an applicator can also be used to apply an ostomy type apparatus to other stomas in the human body that may require the elimination of waste in a sanitary manner.

### SUMMARY OF THE INVENTION

A first object of this invention is to provide a method of applying an ostomy adapter to a stomal opening so that the adhesive-equipped skin barrier, between the apparatus and the skin of the user, makes a firm, safe, secure, comfortable and sanitary seal with the outer abdominal (or other location) wall of the user.

A second object of the present invention is to provide the applicator that will allow the method to be practiced.

These objects will be met by an applicator that is a simple mechanical device that allows a uniform pressure to be exerted against the outer surface of the ostomy adapter, so that the pressure is uniformly and simultaneously transmitted to the entire adhesive surface of the skin barrier on the bottom (underside) surface of the ostomy adapter, thereby producing a firm, safe, secure, comfortable and sanitary seal. The applicator may be a separate device that is conformable to a variety of commercially available ostomy apparatuses. Alternatively it may be shaped to fit specific commercial brands, such as the Esteem™ Synergy brand mentioned above. It may be fashioned of plastics, or any similar hard material that can be molded or machined and washed and rendered sterile for repeated use. Alternatively, it may be manufactured of inexpensive materials such as pressed board that are disposable. It may be equally advantageous to form the applicator as a part of the ostomy adapter itself, so that it can be stripped off and disposed of after use.

### BRIEF DESCRIPTION OF THE DRAWINGS

FIG. 1(a) is a schematic front view and FIG. 1(b) is a schematic side view illustration of the outer cylinder of an embodiment of the ostomy applicator.

FIG. 2(a) is a schematic front view and FIG. 2(b) is a schematic side view of an inner cylinder of the ostomy applicator suitable for insertion into the outer cylinder of FIG. 1 and having a 1.00 in. inner diameter.

FIG. 3(a) is a schematic front view and FIG. 3(b) is a schematic side view of an alternate inner cylinder of the ostomy applicator of FIG. 1, the inner cylinder having a ⅞ in. inner diameter.

FIG. 4 is a schematic plan view of a typical prior art ostomy adapter to which the applicator will be applied.

FIG. 5 is a schematic side, cross-sectional view of the applicator formed by combining the cylinders of FIG. 1 and FIG. 2 (or FIG. 1 and FIG. 3), being applied to the ostomy adapter of FIG. 4.

### DESCRIPTION OF THE PREFERRED EMBODIMENTS

The preferred embodiment of the present invention provides a method of applying an ostomy adapter to the abdominal wall of a user of an ostomy apparatus so that the adhesive region of the skin barrier layer on the underside of the adapter forms a firm, safe, secure, comfortable and sanitary seal against the abdominal wall. The method of application, according to this invention, includes the use of an applicator formed as two axially symmetric concentric cylinders, wherein an annular inner cylinder fits snugly but movably within an annular outer cylinder and is constrained by a set screw to move linearly and remain engaged to the outer cylinder. Each cylinder has a front face that will contact the ostomy adapter and a rear face that will generally contact the hands of the user and against which the user will push to create the necessary pressure. The front face will typically be patterned to fit a corresponding patterning of the ostomy adapter. The movement of the inner cylinder allows the applicator and adapter to conform accurately to bodily shape irregularities and thereby to assure a uniform pressure against the adhesive layer regardless of the shape of the user. During use, both cylinders press against the ostomy apparatus to create a uniform pressure.

The role of the applicator is to provide a uniform pressure against the ostomy adapter and thereby to the outer surface of the skin barrier so that the pressure is transmitted to the adhesive region uniformly and simultaneously around the entire region. The uniformity and simultaneity is easily created in a manner not possible using only the hands without such an applicator. Moreover, the uniformity and simultaneity of pressure can be applied in a reproducible and repeatable fashion whenever and wherever on the body such an ostomy apparatus is applied.

The applicator is formed of an outer, axially symmetric annular cylinder having an outer diameter and an inner diameter, into which is inserted a concentric, linearly co-moving inner annular cylinder. A set screw passing radially through a threaded hole in the outer cylinder engages a slot in the outside of the inner annular cylinder insuring that its motion will be linear and that it will not become disengaged from the outer cylinder and fall out. Hereinafter, where the inner cylinder is described as being engaged with the outer cylinder, what is meant is that the motion of the inner cylinder will be linear relative to the outer cylinder will not be allowed to fall free of the outer cylinder. It is noted that the material of the applicator may be any hard plastic that can be shaped or molded or any other hard formable material that can be cleaned and sterilized if necessary. A hard polypropylene was used to create the present embodiment. Furthermore, the dimensions and surface configurations of the inner cylinder can be designed to fit virtually any skin barrier currently in use. Notwithstanding the exemplary use of a polypropylene material to form a version of the applicator, the objects of the applicator may be realized using disposable materials such as pressed board or other composites. Such materials may allow the applicator to be formed integral with the ostomy adapter itself, whereupon it may be used to exert the requisite pressure and then removed so that the ostomy adapter may accept the ostomy pouch.

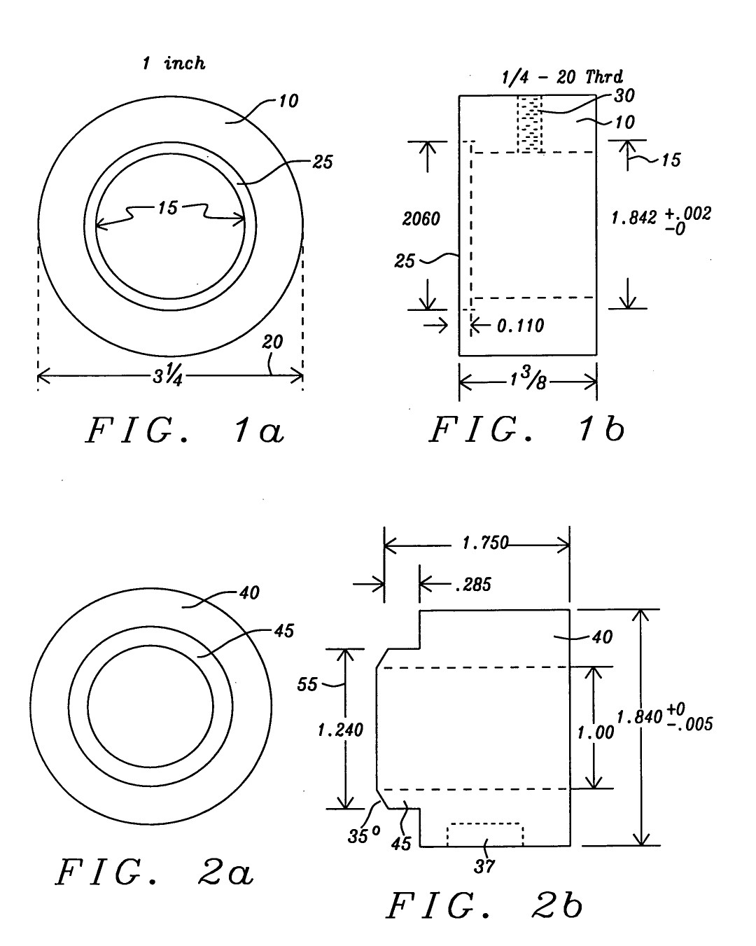

Referring first to FIG. 1(a) there is shown a view of a front face of a preferred embodiment of an outer cylinder (10) within which an inner cylinder (see FIG. 2) will move. The outer cylinder is annular and axially symmetric, having an inner diameter (15) of between approximately 1.840 and 1.845 inches and an outer diameter (20) of approximately 3.25 inches. The front face of the cylinder has an annular recessed region (25) of radius approximately 2.060 inches, symmetrically disposed about the inner diameter and extending inward to a depth of approximately 0.110 inches, as will be seen more clearly in FIG. 1(b). This recessed region is part of a patterning that allows the cylinder to make firm contact with an ostomy adapter. It is understood that different ostomy adapters might require variations in patterning of the cylinders.

Referring next to FIG. 1(b) there is shown a schematic side view of the outer concentric cylinder (10) of FIG. 1(a). The length of the cylinder is approximately 1.375 inches. A 0.25 inch threaded set screw hole (30) radially penetrates the cylinder. A set screw passing through the hole (not shown) will engage a corresponding channel in an inner cylinder to insure linear motion and to retain engagement between the inner and outer cylinders. It is noted that if this cylinder were to be formed of a disposable material or to be made a part of an ostomy apparatus, its length could be reduced.

Referring now to FIG. 2(a) there is shown a schematic front view of an inner cylinder (40) that will fit movably into the inner diameter opening ((15) in FIG. 1(b)) of the outer annular cylinder. The cylinder has an outer diameter of between approximately 1.840 and 1.845 inches and should fit tightly but be linearly moveable within the outer cylinder. The face of the cylinder shown in this view is the surface that will contact the ostomy adapter and provide an optimal fit to corresponding surface structures in the adapter.

Referring to FIG. 2(b) there is shown a schematic side view of the cylinder of FIG. 2(a). As is seen in the side view, an annular ring (45) extends outward from the front face of the cylinder by approximately 0.285 inches. The outer surface of this annular ring is beveled by an angle of approximately 35°. As will be shown in FIG. 5, this outer annular ring will enable the applicator to engage the surface structure of the ostomy apparatus while the applicator is being used. The outer diameter (55) of this outer surface is approximately 1.240 inches. As is seen in the figure, the annular cylinder has an inner diameter (60) of approximately 1.00 inches. As can also be seen in the figure, the overall length of the cylinder (65) is approximately 1.750 inches. A slot (37) in the side of the cylinder of exemplary depth ¼ inch, will engage a set screw (not shown here) to allow only a relative linear motion between the inner and outer cylinders with no rotation and to prohibit disengagement of the inner cylinder from the outer cylinder. It is to be noted that the annular ring in this exemplary inner annular cylinder is design to provide an optimal fit to a commonly used ostomy adapter (see above). Other ostomy adapters may require surface structures on the face of the inner annular cylinder that provide optimal fits to them. Alternatively, a generic set of cylinders may be shaped so as to provide fits to a wide range of ostomy adapters.

Referring now to FIG. 3(a) there is shown a schematic front view of an alternative inner cylinder designed to be used in conjunction with another version of an ostomy adapter that could be applied to a bladder stoma. This cylinder (80) has the same outer diameter (85) of between approximately 1.840 and 1.845 inches required to fit snugly but moveably linearly into the outer cylinder of FIG. 1. This cylinder has an inner diameter (90) of approximately 0.875 inches that does not extend completely through the cylinder, but extends rearward from the front face of the cylinder whereupon it terminates at a smaller cylindrical opening (88) that extends to the rear face of the cylinder. The diameter of the smaller opening (88) is not critical and can be determined so as to snugly engage a drain tube (not shown), which is often required for bladder ostomies that may produce significant drainage even while the ostomy apparatus is being applied.

Referring to FIG. 3(b) there is shown schematically a side view of the cylinder of FIG. 3(a) showing that a first and a second annular ring (100) and (105) extend forward from the cylinder to create a stepped surface. The first annular ring, with the largest outside diameter (100), has an outside diameter of approximately 1.240 inches and extends by approximately 0.165 inches from the outer face of the inner cylinder (80). The second annular projection with the smaller outside diameter (105) has a diameter of approximately 1.125 inches and extends from the first annular ring by approximately 0.135 inches. The overall extension of the two projections is approximately 0.300 inches. It is noted that the surface topography produced by the stepped annular projections (100) and (105) are indicated here only as exemplary structures that were found appropriate to engage a particular urostomy adapter. It is envisioned that other surface structures might be formed to fit other ostomy adapters.

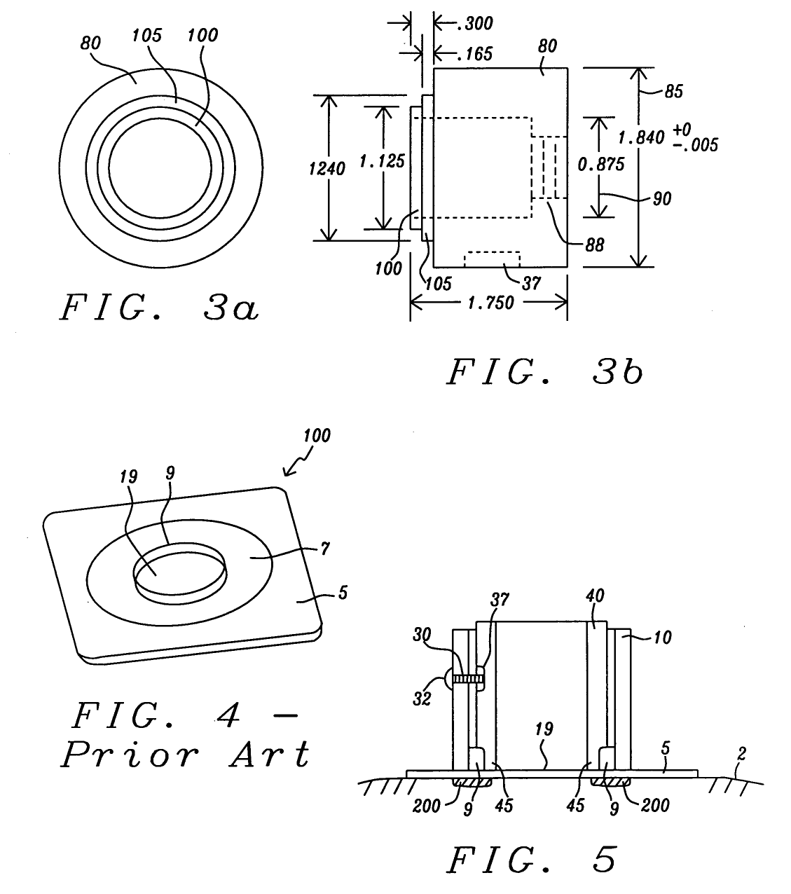

Referring next to FIG. 4, there is shown a schematic illustration of an exemplary, generic, ostomy adapter (100) of the type to which the present invention can be advantageously applied. The perspective view illustrated in the figure shows the outer surface of the adapter to which an ostomy bag (not shown) will ultimately be affixed. The adapter (100) includes a base (5) with a flanged (9) annular connector (7). The flanged (9) circular opening (19) of the connector (7) would normally be positioned over the stoma of the user. On the bottom surface of the adapter base (5), on a region surrounding the opening (19) that is substantially coincident with the connector (7), there is the skin shield. The skin shield is coated with an annular region of adhesive that is substantially beneath the annular connector (7). Once the adhesive is affixed to the user, by means of the applied pressure of the present applicator, an ostomy bag (not shown) would be fastened to the connector (7).

Referring next to FIG. 5, there is shown, schematically, a side cross-sectional view of the ostomy applicator of the present invention (combined outer cylinder of FIG. 1 and inner cylinder of FIG. 2) placed over the ostomy adapter of FIG. 4 preparatory to using the applicator to secure the adapter to the skin of the user. The applicator has been constructed by inserting the inner cylinder of FIG. 2 (shown here in cross-sectional view as (40)) into the outer cylinder of FIG. 1 (shown here in cross-sectional view as (10)) and a set screw (32) has passed through the hole (30) in the outer cylinder to engage the slot (37) in the inner cylinder wall (40) to insure linear motion and maintain engagement of the cylinders. The applicator has been placed over the adapter flange (9), thereby positioning the applicator over the adhesive portion (200) (shown shaded) of the skin barrier beneath the base (5) in a position to produce the secure seal to the skin (2) that is the object of the invention. Annular ring (45) of the inner cylinder of the applicator fits into the flanged (9) opening (19) of the skin barrier of the ostomy adapter. The outer cylinder and the inner cylinder can now both be pushed down against the skin barrier to exert a uniform pressure over the adapter that will be transmitted to the adhesive underside to create the desired firm, safe, secure, comfortable and sanitary seal. It is noted that the applicator as shown in this figure could be formed as a part of the ostomy adapter instead of being a separate device. In the case of an integral applicator/adapter device, the same structure of FIG. 5 would describe the combination.

As is finally understood by a person skilled in the art, the preferred embodiments of the present invention are illustrative of the present invention rather than limiting of the present invention. Revisions and modifications may be made to methods, materials, structures and dimensions employed in providing and using an applicator for a skin barrier of an ostomy apparatus while still providing and using such a device in accord with the spirit and scope of the present invention as defined by the appended claims.

What is claimed is:

1. A method for adhesively affixing an ostomy adapter to the bodily surface skin of a user thereof comprising:
positioning the ostomy adapter over a stoma whereby an adhesive layer of a skin barrier on a bottom surface of said ostomy apparatus annularly surrounds said stoma and contacts the skin of said user;
placing an applicator against a top surface of said ostomy adapter so that an inner annular cylinder of said applicator engages an opening in said adapter, wherein said opening is positioned over said stoma; then
exerting a downward force on said inner annular cylinder and simultaneously exerting a downward force on an outer annular cylinder within which said inner cylinder is engaged and moves linearly; whereby
said adapter conforms to said bodily surface and said outer annular cylinder and said inner cylinder both exert a uniform pressure simultaneously on said adhesive layer, affixing it thereby to said skin.

2. The method of claim 1 further including attaching a drain tube to an opening in said inner annular cylinder to permit drainage of bodily waste while said adapter is being adhesively affixed.

3. The method of claim 1 wherein said applicator comprises:
an annular outer cylinder; and
an annular inner cylinder; wherein
said annular inner cylinder fits concentrically within said annular outer cylinder; and
said annular inner cylinder slides linearly within said annular outer cylinder and remains engaged therewith by means of a set screw passing through said outer cylinder and engaging a slot in said inner cylinder.

4. The method of claim 3 wherein said inner annular cylinder includes an opening for a drain tube.

5. The method of claim 3 wherein surface structures on said applicator engage corresponding structures on said ostomy adapter.

6. An applicator for an ostomy adapter comprising:
an annular outer cylinder; and
an annular inner cylinder; wherein
said annular inner cylinder fits concentrically within said annular outer cylinder; and
said annular inner cylinder slides linearly within said annular outer cylinder and remains engaged therewith by means of a set screw passing through said outer cylinder and engaging a slot in said inner cylinder.

7. The applicator of claim 6 wherein said annular inner cylinder includes an opening for a drain tube.

8. The applicator of claim 6 wherein said annular inner cylinder includes a projecting annular ring that engages an opening in a skin barrier.

9. The applicator of claim 6 wherein surface structures on faces of said inner annular cylinder engage corresponding structures on an ostomy adapter and allow said adapter to conform to an abdominal wall while said outer annular cylinder and said inner annular cylinder exert a uniform pressure on said ostomy adapter.

10. The applicator of claim 6 wherein said annular inner cylinder and said annular outer cylinder are formed of hard plastic.

11. The applicator of claim 6 wherein said annular inner cylinder and said annular outer cylinder are formed as part of said ostomy adapter.

12. The applicator of claim 6 wherein said outer cylinder has an outer diameter of approximately 3.25 inches, an inner diameter of between approximately 1.840 and 1.845 inches and a length of approximately 1.375 inches.

13. The applicator of claim 12 wherein said outer cylinder has a front face and a rear face and wherein said front face includes an annular recess of diameter approximately 2.060 inches, symmetrically disposed about said inner diameter, wherein said recess extends inward, in a length direction, to a depth of approximately 0.110 inches.

14. The applicator of claim 13 wherein a threaded hole of diameter approximately 0.25 inches extends in a radial direction through said outer cylinder from said outer diameter to said inner diameter.

15. The applicator of claim 14 wherein said inner annular cylinder has an outside diameter of between approximately 1.840 and 1.845 inches, enabling a fit within the inner diameter of said outer cylinder and wherein said inner cylinder has an inside diameter of approximately 1.00 inches and wherein a longitudinal slot is formed in an outer surface of said annular cylinder of sufficient width to accommodate a set screw of diameter approximately 0.25 inches and of sufficient length to maintain engagement of said inner cylinder within said outer cylinder.

16. The applicator of claim 15 wherein a length between a front face and a rear face of said inner annular cylinder is approximately 1.465 inches and wherein a concentric annular ring of outer diameter approximately 1.240 inches and inner diameter approximately 1.00 inches extends outward from said front face for a length of approximately 0.285 inches and wherein said concentric annular ring is beveled to an angle of approximately 35° at an outer edge.

17. The applicator of claim 14 wherein said inner annular cylinder has a length of approximately 1.450 inches an outside diameter of between approximately 1.840 and 1.845 inches, enabling a fit within the inner diameter of said outer cylinder and wherein two concentric annular rings extend outward from a front face of said inner cylinder, wherein a first one of said rings has an outer diameter of approximately 1.240 inches and extends outward from said front face of said inner annular cylinder for a length of approximately 0.165 inches and a second one of said annular rings has an outer diameter of approximately 1.125 inches and extends outward from said first annular ring for a length of approximately 0.135 inches.

18. The applicator of claim 17 wherein the inner diameter of said inner annular cylinder is approximately 0.875 inches extending from a front face of said second annular ring for a distance less than 1.750 inches and reduces to a diameter less that 0.875 inches for the remaining distance to the rear face of said inner annular cylinder and wherein a longitudinal slot is formed in an outer surface of said inner annular cylinder of sufficient width to accommodate a set screw of diameter approximately 0.25 inches and of sufficient length to maintain engagement of said inner cylinder within said outer cylinder.
